(19) United States
(12) Patent Application Publication
(10) Pub. No.: US 2007/0104771 A1
(43) Pub. Date: May 10, 2007

(54) Transdermal galantamine delivery system

(76) Inventors: Jay Audett, Mountain View, CA (US); Eli J. Goldman, San Francisco, CA (US); Delphine C. Imbert, Cupertino, CA (US); Diane E. Nedberge, Los Altos, CA (US); Jianye Wen, Palo Alto, CA (US); Allison Luciano, Lebanon, NJ (US); Eric N. Silverberg, Summit, NJ (US); Paul B. Foreman, Somerville, NJ (US); Johan H. Geerke, Los Altos, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/525,696

(22) Filed: Sep. 22, 2006

Related U.S. Application Data
(60) Provisional application No. 60/720,209, filed on Sep. 23, 2005.

(51) Int. Cl. A61K 9/70; A61K 31/55

(52) U.S. Cl. 424/449; 514/214.01

(57) ABSTRACT

A transdermal galantamine delivery system to an individual. The system has a high galantamine loading with suitable permeation enhancers to effect therapeutic flux rate. Acrylate polymeric reservoir with the high galantamine and permeation enhancers dissolved therein provides desirable adhesive characteristics and effective transdermal therapeutic properties for multiple-day delivery.

### CROSS REFERENCE TO RELATED U.S. APPLICATION DATA

The present application is derived from and claims priority to provisional application U.S. Ser. No. 60/720,209, filed Sep. 23, 2005, which is herein incorporated by reference in its entirety.

### TECHNICAL FIELD

This invention relates to a medical patch for transdermal administration of galantamine and to a method of treating a subject by administering galantamine thereto with a medical patch. More particularly, the invention relates to transdermal systems for administration of galantamine with adhesive system having high drug and enhancer tolerance when used in transdermal drug delivery.

### BACKGROUND

Transdermal devices for the delivery of biologically active agents have been used for maintaining health and therapeutically treating a wide variety of ailments. For example, analgesics, steroids, etc., have been delivered with such devices. Such transdermal devices include patches in which a biologically active agent is delivered to the body tissue passively without use of an additional energy source. Many such devices have been described, for example, in U.S. Pat. Nos. 3,598,122, 3,598,123, 4,379,454, 4,286,592, 4,314,557, 4,568,343, and U.S. Application No. 2003002682, all of which are incorporated herein by reference.

A transdermal patch is typically a small adhesive bandage that contains the drug to be delivered. A simple type of such transdermal patches is an adhesive monolith including a drug-containing reservoir disposed on a backing. The reservoir is typically formed from a pharmaceutically acceptable pressure sensitive adhesive. In some cases, the reservoir can be formed from a non-adhesive material, the skin-contacting surface of which is provided with a thin layer of a suitable adhesive. The rate at which the drug is administered to the patient from these patches can vary due to normal person-to-person and skin site-to-skin site variations in the permeability of skin to the drug.

Some patches can be multilaminate and include a drug release-rate controlling membrane disposed between a drug reservoir and the skin-contacting adhesive. This membrane, by decreasing the in vitro release rate of drug from the patch, is used to reduce the effects of variations in skin permeability.

Although the transdermal delivery of therapeutic agents has been the subject of intense research and development for over 30 years, only a relatively small number of drug molecules are suitable for transdermal delivery due to the fact that human skin is an excellent barrier. Various techniques have been explored to enhance the permeation of drug molecules that are not otherwise suitable for transdermal delivery. Of these techniques, chemical enhancement is the most established and is currently employed commercially. Pressure sensitive adhesives, such as acrylic adhesives, are used in most transdermal drug delivery devices as a means of providing intimate contact between the drug delivery device and the skin. The use of enhancers, especially at high concentrations, usually has a significant impact on the properties of pressure sensitive adhesives, such as cohesive strength, adhesive flow, tackiness and adhesion strength. Therefore, pressure sensitive adhesives have to be designed in a way that they can provide the needed performance in the presence of enhancer.

Such a need is especially keen for drugs such as galantamine, which is hard to deliver in doses high enough for therapy for ailments such as Alzheimer's disease or dementia. Galantamine, also called galanthamine, has the structure C₁₇H₂₁NO₃, and is chemically named 4a, 5, 9, 10, 11, 12-Hexahydro-3-methoxy-11-methyl-6H-benzofuro (3a, 3, 2-ef) (2) benzazepin-6-ol. The preparation and pharmacological activity of galantamine have been described in U.S. Pat. Nos. 5,877,172; 6,194,404; 6,335,328; 6,573,376; and 6,617,452. Further, transdermal delivery of galantamine has been mentioned in U.S. Pat. Nos. 5,700,480; 5,932,238; as well as U.S. Patent Publication No. 20040192683. Galantamine is one of the reversibly acting cholinesterase inhibitors. It is reported to have effects similar to those of physostigmine and neostigmine but presents a lower risk of toxicity as physostigmine or neostigmine. Galantamine has been reported to be useful for treatment of the narrow-angle glaucoma and, as antidote after curare applications, treatment of dementia, Alzheimer's disease and the treatment of alcohol dependence.

However, for transdermal applications, drug loading and flux have been low. For example, a drug loading of 10 wt % cannot support a multi-day system without the introduction of a secondary drug reservoir or very thick adhesive layer. Prior art references on transdermal galantamine delivery do not teach systems that permits high galantamine solubility and flux rates. For example, U.S. Pat. No. 5,700,480 describes a flux that if delivered from a reasonably small patch would be low for effective therapy of Alzheimer's disease. The flux reported there was on mice skin, which tends to have higher permeability than human skin. For delivery to human, better design to improve galantamine permeation will be required. Thus, a transdermal galantamine delivery device with good drug loading and sufficient flux is needed for effective therapy of Alzheimer's disease from a reasonably sized patch. There continues to be a need for improved delivery of galantamine, especially sustained delivery over a period of time.

### SUMMARY

This invention provides transdermal galantamine delivery devices and formulations that deliver galantamine base at a therapeutically effective level. The formulations have low irritation potential and contain sufficient drug and enhancer to support multi-day delivery at a reasonable adhesive thickness. The incorporation of the ingredients, such as permeation enhancers, of the formulations provide enhanced rheological properties suitable for transdermal delivery. The therapeutic dose required for treating Alzheimer's disease with galantamine is between 16 to 24 mg per day of the oral dose. Based on an oral bioavailability of 90%, this is equivalent to a transdermal flux of 14.4 to 21.6 mg per day, e.g., at a rate of 12.5 to 18.75 μg/cm²hr with a system area of 48 cm², and equivalent to about 7 to 13 μg/cm²hr flux for a 80 cm² patch. For therapeutic results, the delivery requirements of galantamine are quite high and cannot be achieved without suitable permeation enhancers. In one aspect, the selected permeation enhancer(s) according to the present invention facilitate the flux needed for therapy. The present invention allows transdermal delivery of galantamine with high loading of galantamine and permeation enhancer(s) dissolved in acrylate polymer.

In one aspect, a transdermal delivery device is provided with high enough galantamine content, preferably completely dissolved into a drug reservoir matrix. In another aspect, a transdermal delivery device is provided with an acrylate polymeric material in the drug reservoir matrix and yet resulting in a device with desirable rheological properties.

Many transdermal systems under development today incorporate the drug and permeation enhancers directly into the pressure sensitive adhesive. These systems are thinner, more comfortable to wear, and much easier to manufacture, but require sophisticated pressure sensitive adhesives to be effective. In particular, the adhesive must have very high drug and enhancer solubility while maintaining the adhesive properties of the system. Finding such an adhesive is quite difficult and is usually the difference between a successful product and a product that never makes it market. One of the more valuable aspects of this invention is the base polymer from which the formulations were developed. The preferred acrylate base polymer has a high polar functionality (e.g., acid and hydroxyl functional groups), enabling high drug loading in the adhesive system. Of course, the longer lasting the patch for multiple day delivery, the more the drug and permeation enhancers will be needed. The drug reservoir with increased loading of the present invention allows for 3-day, 7-day delivery at a reasonable adhesive thickness.

In a preferred aspect, it is possible to load drug and/or enhancer into the polymer composition to a high concentration, e.g., at greater than 20 dry weight %, greater than 30 dry weight % (or solids wt %), even up to 40-50 wt %, and still provide adequate adhesion and Theological characteristics for pressure sensitive adhesive (PSA) application. With sufficient loadings of permeation enhancers in such formulations, sustained high rates of drug delivery can be achieved. With adequate adhesive properties, the resulting reservoir with sufficient drug loading and permeation enhancers can be used to achieve effective therapeutic results. In such embodiments, prior to incorporation of drugs and ingredients, the polymeric materials are not suitable PSAs “as is” because of the stiffness of the polymer and insufficient adhesiveness or tackiness. These polymeric materials become adhesive and have the desired PSA characteristics after incorporating drugs, permeation enhancer and optionally other ingredients in suitable quantities. Such polymeric materials, which are not suitable as a PSA as is (prior to incorporation of drugs and ingredients) but will have the desired PSA characteristics after incorporating drugs and/or other ingredients, can be called “proadhesive” herein. It has been discovered that by increasing the glass transition temperature of the acrylate polymer using the ratio of soft monomer and hard monomer, it is possible to load enhancer concentrations into the proadhesive acrylate polymer at a high weight percent to obtain a formulation and still achieve desirable adhesive characteristics.

The present invention provides a method and a device for transdermal delivery of galantamine for therapeutic effects, especially delivery of the galantamine to a subject through skin or other body surface that is accessible from exterior without using endoscopic devices. Once applied on an individual's body surface, the device can stay adhesively to the body surface over an extended period of time. The transdermal delivery of galantamine may result in lower adverse events than what is seen with oral delivery. Further, a transdermal patch will allow a more steady sustained delivery than doses taken orally at time intervals hours apart. The transdermal form of the drug could allow use in the elderly patient population, especially those that have neurological ailments and would have difficulty in taking oral medication at regular intervals. This invention allows for the transdermal delivery of a therapeutic dose of galantamine (about 14.4 to 21.6 mg per day) from a thin, flexible, user-friendly patch about 40 to 125 cm² in size. It also provides us with a method to load enough galantamine into the drug reservoir of the transdermal patch that can be worn for an extensive period of time, such as 3, even 7 days. Patches that can be used for such extensive periods of time would increase patient compliance and would reduce care-giver's burden.

In one aspect, the present invention provides a system for transdermal delivery of galantamine. In another aspect, the present invention to provide a transdermal galantamine delivery system with improved enhancer and galantamine loading, as well as acceptable rheological and adhesive properties.

The transdermal formulation of galantamine will address some of the challenges to providing optimal galantamine therapy. A 7-day transdermal delivery system would likely reduce caregiver burden and improve dosing compliance. Furthermore, transdermal delivery of drug should result in less gastrointestinal exposure compared to oral administration and could decrease the incidence of gastrointestinal side effects associated with peripheral cholinergic stimulation. Transdermal flux rates which produce gradually increasing plasma levels over several days may reduce the need for dosing titration and simplify the dosing regimen. An ability to achieve and tolerate higher galantamine levels or more rapid dose titration would be expected to result in greater efficacy, earlier onset of symptomatic improvement, or both.

### BRIEF DESCRIPTION OF THE DRAWINGS

FIG. 1 illustrates a cross-section through a schematic, perspective view of one embodiment of a transdermal therapeutic system according to the present invention.

FIG. 2 illustrates a cross-section view through another embodiment of a transdermal therapeutic system of this invention.

### DETAILED DESCRIPTION

The present invention relates to transdermal delivery of galantamine or a salt thereof, especially the uncharged base form of galantamine, with the help of permeation enhancers for loading adequate amount of galantamine. The present invention relates especially to galantamine that is delivered with the use of an acrylate polymer material that after incorporating galantamine and other ingredients therein can act as a pressure sensitive adhesive (PSA) and maintain the transdermal delivery system on a body surface of an individual.

The present invention has utility in connection with the delivery of galantamine or analogs or derivatives thereof to an individual in need the galantamine treatment through body surfaces and membranes, including skin. Galantamine derivatives and analogs are known in the art. It is noted that galantamine analogs and derivatives that have solubilities better or comparable to that of galantamine base can be incorporated into the device with or in place of galantamine base. Galantamine derivative and analogs have been disclosed in for example, the following, U.S. Pat. Nos. 5,958,903; 6,018,043; 6,093,815; 6,184,004; 6,316,439; 6,323,195; 6,323,196; US Publication 20050065338, and European Patent Application No. EP1458724A, which are incorporated by reference in their entireties herein.

A suitable transdermal delivery patch according to the present invention is about 5-125 cm² in area, and preferably about 20 to 80 cm² in area, especially about 20 cm to 60 cm² in area. For effective therapy, for example, the delivery of about 14.4 to 21.6 mg daily dose, a transdermal galantamine flux in a range of 12.5-18.8 μg/cm²-hr, for a system area of about 48 cm² is applicable. For a 80 cm² patch, about 7 to 13 μg/cm²hr flux is applicable. For a seven day patch, a drug loading in excess of 15 wt % and a drug reservoir thickness of less than about 0.25 mm (10 mil) is preferred. If a semi-weekly patch is used, the thickness can be reduced. The wt % drug loading can be reduced if a thicker drug reservoir is used. When a prolonged therapeutic effect is desired, an old patch is removed and a fresh one applied to a new location. In such cases, blood levels will remain reasonably constant.

The dissolved galantamine content on solids in the drug reservoir matrix can be above 10 wt %, preferably above 15 wt %, more preferably from 15 wt % to 35 wt %, more preferably above 20 wt %, more preferably from 20 wt % to 30 wt %. Such galantamine contents are suitable for effecting flux of therapeutic effect for ailments such as Alzheimer's disease or dementia, with a flux of, e.g., greater than 5 μg/cm²-hr, preferably greater than 7 μg/cm²-hr, preferably about 10 μg/cm²-hr to 15 μg/cm²-hr for a 3 day patch, preferably a 7 day patch.

Traditionally a transdermal drug delivery system was formulated with a pressure sensitive adhesive that has a glass transition temperature (T_{g}) in the range of −40° C. to −10° C. According to the present invention, a useful reservoir material is acrylate polymer. In one aspect of the present invention, a preferred starting acrylate polymeric material (which can be formulated into an adhesive material having drugs and/or enhancers) preferably has a glass transition temperature (T_{g}) in the range of −20° C. or higher, preferably −15° C. or higher, more preferably −15° C. to 0° C., and even more preferably −10° C. to 0° C.; creep compliance of about 7×10⁻⁵ cm²/dyn (at 3600 second) or below; and modulus G′ of about 8×10⁵ dyn/cm² or above. The polymeric material can be formulated into a transdermal reservoir matrix (including carrier structure) with a combined drug and/or enhancer concentration greater than 30 dry weight percent (wt %), or even greater than 40 dry weight percent. The resulting transdermal adhesive formulation with pharmaceutical agent(s) and/or enhancers will provide excellent adhesion with no cold flow, i.e., with no cold flow of an amount that is noticeable and would affect the normal use of the delivery system. By contrast, the starting proadhesive acrylate polymer has poor adhesive properties because the glass transition temperature is too high. Once plasticized in the transdermal formulation, the glass temperature drops into the pressure sensitive range, about −10 to −40° C., and the resulting creep compliance and storage modulus enables the achievement of good tack, with little or no cold flow. Creep compliance is an important parameter to evaluate cold flow behavior of a pressure sensitive adhesive (PSA). In a transdermal drug delivery system, if the creep compliance is large, the adhesive will have cold flow with time, i.e., the adhesive may lose its shape just because of the weight of the material in the device under gravity.

In describing the present invention, the following terms will be employed, and are intended to be defined as indicated below. As used in this specification and the appended claims, the singular forms “a,” “an” and “the” include plural references unless the text content clearly dictates otherwise.

As used herein, the term “transdermal” refers to the use of skin, mucosa, and/or other body surfaces as a portal for the administration of drugs by topical application of the drug thereto for passage into the systemic circulation.

“Biologically active agent” is to be construed in its broadest sense to mean any material that is intended to produce some biological, beneficial, therapeutic, or other intended effect, such as enhancing permeation, or relief of symptoms of neurological disorder. As used herein, the term “drug” refers to any material that is intended to produce some biological, beneficial, therapeutic, or other intended effect, such as relief of symptoms of a disorder, but not agents (such as permeation enhancers) the primary effect of which is to aid in the delivery of another biologically active agent such as the therapeutic agent transdermally.

As used herein, the term “therapeutically effective” refers to the amount of drug or the rate of drug administration needed to produce the desired therapeutic result. As used herein, the term “permeation enhancement” intends an increase in the permeability of skin to a drug in the presence of a permeation enhancer as compared to permeability of skin to the drug in the absence of a permeation enhancer. A “permeation-enhancing amount” of a permeation-enhancer is an amount of the permeation enhancer sufficient to increase the permeability of the body surface of the drug to deliver the drug at a therapeutically effective rate.

“Acrylate”, “polyacrylate” or “acrylic polymer”, when referring to a polymer for an adhesive or proadhesive, refers to polymer or copolymer of acrylic acid, ester(s) thereof, acrylamide, or acrylonitrile. Unless specified otherwise, it can be a homopolymer, copolymer, or a blend of homopolymers and/or copolymers.

As used in the present invention, “soft” monomers refer to the monomers that have a T_{g} of about −80 to −10° C. after polymerization into homopolymer; “hard” monomers refer to the monomers that have a T_{g} of about 0 to 250° C. after forming homopolymer; and “functional” monomers refer to the monomers that contain hydrogen bonding functional groups such as hydroxyl, carboxyl or amino groups (e.g., alcohols, carboxylic acid, or amines), these polar groups tend to increase the hydrophilicity of the acrylate polymer and increase polar drug solubility.

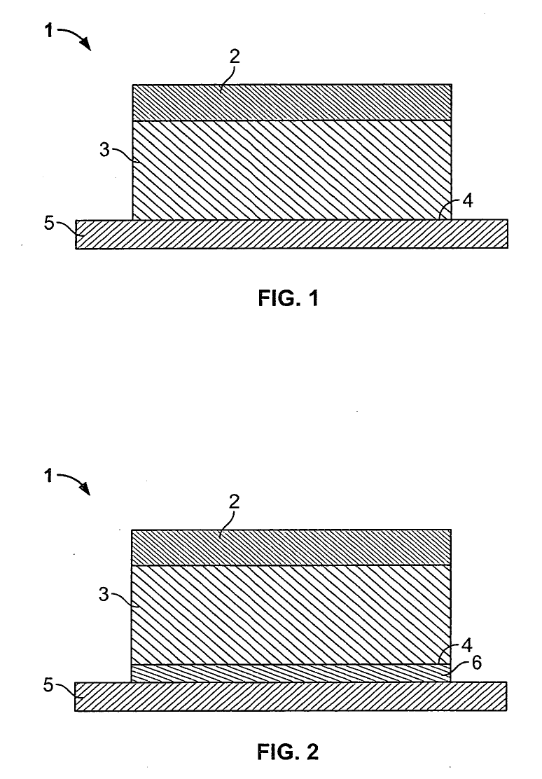

Exemplary transdermal drug delivery systems of the present invention are illustrated by the embodiments shown in FIGS. 1 and 2. As shown in FIGS. 1 and 2, an embodiment of the transdermal monolithic patch 1 according to this invention has a backing layer 2, a drug reservoir 3 disposed on the backing layer 2, and a peelable protective layer 5. In the reservoir 3, which can be a layer, at least the skin-contacting surface 4 is an adhesive. The reservoir is a matrix (carrier) that is suitable for carrying the pharmaceutical agent (or drug) galantamine for transdermal delivery. Preferably, the whole matrix, with drugs and other optional ingredients, is a material that has the desired adhesive properties. The reservoir 3 can be either a single phase polymeric composition or a multiple phase polymeric composition. In a single phase polymeric composition the drug and all other components are present at concentrations no greater than, and preferably less than, their saturation concentrations in the reservoir 3. This produces a composition in which all components are dissolved. The reservoir 3 is formed using a pharmaceutically acceptable polymeric material that can provide acceptable adhesion for application to the body surface. In a multiple phase polymeric composition, at least one component, for example, a therapeutic drug, is present in amount more than the saturation concentration. In some embodiments, more than one component, e.g., a drug and a permeation enhancer, is present in amounts above saturation concentration. In the embodiment shown in FIG. 1, the adhesive acts as the reservoir and includes a drug.

In the embodiment shown in FIG. 2, the reservoir 3 is formed from a material that does not have adequate adhesive properties if without drug or permeation enhancer. In this embodiment of a monolithic patch 1, the skin-contacting surface of the reservoir 4 may be formulated with a thin adhesive coating 6. The reservoir 3 may be a single phase polymeric composition or a multiple phase polymeric composition as described earlier, except that it may not contain an adhesive with adequate adhesive bonding property for the body surface (e.g.) skin. The adhesive coating can contain the drug and permeation enhancer, as well as other ingredients.

The drug reservoir 3 is disposed on the backing layer 2. At least the skin-contacting surface of the reservoir is adhesive. As mentioned, the skin-contacting surface can have the structure of a layer of adhesive. The reservoir 3 may be formed from drug (or biological active agent) reservoir materials as known in the art. For example, the drug reservoir is formed from a polymeric material in which the drug has reasonable solubility for the drug to be delivered within the desired range, such as, a polyurethane, ethylene/vinyl acetate copolymer (EVA), acrylate, styrenic block copolymer, and the like. In preferred embodiments, the reservoir 3 is formed from a pharmaceutically acceptable adhesive or proadhesive, preferably acrylate copolymer-based, as described in greater detail below. The drug reservoir or the matrix layer can have a thickness of about 1-10 mils (0.025-0.25 mm), preferably about 2-5 mils (0.05-0.12 mm), more preferably about 2-3 mils (0.05-0.075 mm).

Preferred materials for making the adhesive reservoir or adhesive coating, and especially for making proadhesives according to the present invention include acrylates, which can be a copolymer of various monomers ((i) “soft” monomer, (ii) “hard” monomer, and optionally (iii) “functional” monomer) or blends including such copolymers. The acrylates (acrylic polymers) can be composed of a copolymer (e.g., a terpolymer, i.e., made with three monomers; or a tetrapolymer, i.e., made with four monomers) including at least two or more exemplary components selected from the group including acrylic acids, alkyl acrylates, methacrylates, copolymerizable secondary monomers or monomers with functional groups. Functional monomers are often used to adjust drug solubility, polymer cohesive strength, or polymer hydrophilicity.

Examples of functional monomers are acids, e.g., acrylic acid, methacrylic acid and hydroxy-containing monomers such as hydroxyethyl acrylate, hydroxypropyl acrylate, acrylamides or methacrylamides that contain amino group and amino alcohols with amino group protected. Functional groups, such as acid and hydroxyl groups can also help to increase the solubility of basic ingredients (e.g., drugs) in the polymeric material. Additional useful “soft” and “hard” monomers include, but are not limited to, methoxyethyl acrylate, ethyl acrylate, butyl acrylate, butyl methacrylate, hexyl acrylate, hexyl methacrylate, 2-ethylbutyl acrylate, 2-ethylbutyl methacrylate, isooctyl acrylate, isooctyl methacrylate, 2-ethylhexyl acrylate, 2-ethylhexyl methacrylate, decyl acrylate, decyl methacrylate, dodecyl acrylate, dodecyl methacrylate, tridecyl acrylate, tridecyl methacrylate, acrylonitrile, methoxyethyl acrylate, methoxyethyl methacrylate, and the like. Additional examples of acrylic adhesive monomers suitable in the practice of the invention are described in Satas, “Acrylic Adhesives,” Handbook of pressure-Sensitive Adhesive Technology, 2nd ed., pp. 396-456 (D. Satas, ed.), Van Nostrand Reinhold, New York (1989). Examples of acrylic adhesives are commercially available from National Starch and Chemical Company, Bridgewater, N.J.

The acrylate polymers can include cross-linked and non-cross-linked polymers. The polymers can be cross-linked by known methods to provide the desired polymers. However, cross-linking is hard to control and may result in polymeric materials that are too stiff or too soft. According to the present invention, it is preferred that the polymeric material for incorporation of drugs and other ingredients to be polymers without crosslinking and no cross-linking agent is used in forming the polymeric material. It is further preferred that the monomers do not self cross-link during polymerization. In the present invention, it was found that, instead of cross-linking to form a matrix adhesive with desired PSA properties for incorporating drugs and enhancers, good control of the PSA properties can be achieved by selecting polymeric materials that are too stiff prior to incorporation of drugs and other ingredients and subsequently incorporating such drugs and ingredients. It has been found that an acrylate polymer composition with a creep compliance (J) of 7×10⁻⁵ cm²/dyn or below and elastic modulus G′ of 8×10⁵ dyn/cm² or above, although too stiff as a PSA as is, after formulating with drug or enhancer or a combination thereof at a relative high concentration will achieve the desirable adhesive properties. The plasticizing or tackifying effect of the drug(s) and/or other ingredients on the polymeric material provides a means to achieve the desired adhesive properties in the reservoir.

Acrylate polymers, when the main monomer of which has the general formula CH₂═CH—COOR, are particularly useful as proadhesives. Typical main monomers are normally alkyl acrylates of 4 to 1 carbon atoms, preferably 4-10 carbons. Useful alkyl acrylates include ethyl acrylate, butyl acrylate, amyl acrylate, hexyl acrylate, 2-ethylhexyl acrylate, octyl acrylate, isooctyl acrylate, decyl acrylate, dodecyl acrylates, with 2-ethylhexyl acrylate, butyl acrylate, and iso-octyl acrylate being preferred. Such “soft” monomers if polymerized into homopolymer generally have a T_{g} of less than about 0° C., preferably about −10° C. to −80° C., preferably about −20° C. to −80° C. Preferably, they are present in an amount of about 10 to 70 wt % (i.e., dry weight % or solids wt %), more preferably no more than about 60% by weight, more preferably no more than about 50 wt % of the total monomer weight and more preferably about 40 to 50 wt %. As used herein, when a monomer is said to be present in the acrylate polymer at a certain percentage, it is meant that the monomer has been polymerized in the acrylate polymer at that percentage of polymerization monomer ingredients.

“Hard” modifying monomers are mainly used to modify the adhesive properties, mainly glass transition temperature (e.g., to increase the T_{g} and to make the resulting polymer stiffer at room temperature), to meet various application requirements. A hard monomer, if polymerized into homopolymer, has a T_{g} of about 0 to 250° C., preferably about 20 to 250° C., more preferably in the range of about 30 to 150° C. (for convenience, this is referred to as the “homopolymer T_{g}” herein). The hard monomer component (or content in the polymer) is present in an amount of about 10 wt % or more, preferably in the range of about 30 to 60 wt %, preferably about 35 to 60 wt %, more preferably about 40 to 60 wt %, even more preferably about 40 to 50 wt % in the polymerization. Examples of hard modifying monomers are methyl acrylate, vinyl acetate, methyl methacrylate, isobutyl methacrylate, vinyl pyrrolidone, substituted acrylamides or methacrylamides. Homopolymers of these monomers generally have higher glass transition temperature than homopolymers of the soft monomers.

Certain nitrogen containing monomers can be included in the polymerization to raise the T_{g}. These include N-substituted acrylamides or methacrylamides, e.g., N-vinyl pyrrolidone, N-vinyl caprolactam, N-tertiary octyl acrylamide (t-octyl acrylamide), dimethyl acrylamide, diacetone acrylamide, N-tertiary butyl acrylamide (t-butyl acrylamide and N-isopropyl acrylamide (i-propyl acrylamide). Further examples of monomers that can be used in polymerization to modify and raise the T_{g} of the polymer include cyanoethylacrylates, N-vinyl acetamide, N-vinyl formamide, glycidyl methacrylate and allyl glycidyl ether.

Functional monomers can be used to either provide needed functionality for solubilizing agents in the polyacrylate or improve cohesive properties. Examples of functional monomers are organic acids, e.g., acrylic acid, methacrylic acid, and hydroxyl-containing monomers such as hydroxyethyl acrylate. Preferred functional monomers when incorporated into the polymer result in acid groups, i.e., —COOH, hydroxyl groups, i.e., —OH, or amino groups in the polymer for affecting the solubility of basic agents such as basic drugs. Examples of hydroxy functional monomers include hydroxyethyl acrylate, hydroxypropyl acrylate, hydroxyethyl methacrylate and hydroxypropyl methacrylate. The hydroxyl groups can be primary, secondary or tertiary hydroxyl. In some cases, the acrylate polymer can includes at least one non-primary hydroxyl functional monomer component to provide orientation of the functional group in the polymer. Suitable non-primary hydroxyl functional monomers are secondary hydroxyl functional monomers such as hydroxypropyl acrylate. Useful carboxylic acid monomers to provide the functional group preferably contain from about 3 to about 6 carbon atoms and include, among others, acrylic acid, methacrylic acid, itaconic acid, and the like. Acrylic acid, methacrylic acid and mixtures thereof are particularly preferred as acids.

A functional monomer can also be a hard monomer, if its homopolymer has the high T_{g}. Such functional monomers that can also function as hard monomers include, e.g., hydroxyethyl acrylate, hydroxypropyl acrylate, acrylic acid, dimethylacrylamide, dimethylaminoethyl methacrylate, tert-butylaminoethyl methacrylate, methoxyethyl methacrylate, and the like.

The functional monomer(s) are preferably present in the acrylate polymer in an amount of about at least 5 wt %, preferably at least 10 wt %, preferably 10 to 40 wt %, more preferably about 10 to 30 wt %, more preferably about 10 to 20 wt %, even more preferably 10 to 15 wt %, even though some of the functional monomer(s) may be hard monomers. Examples of preferred functional monomer component include acrylic acid and hydroxyethyl acrylate, acrylamides or methacrylamides that contain amino group and amino alcohols with amino group protected. One of the applications of using functional monomers is to make a polar proadhesive having higher enhancer tolerance, in that, for example, the resulting PSA with the enhancers and/or drug will not phase separate or have excessive cold flow.

In certain embodiments, the hard monomer(s) that are not also functional monomer can constitute about 10 to 60 wt %, preferably about 40 to 60 wt % of the acrylate monomer, especially in cases in which no acidic functional hard monomer and less than about 20 wt % of hydroxyl functional hard monomer are included in the acrylate polymer. In other embodiments, the hard monomer(s) that are not also functional monomer can constitute about 5 to 15 wt %, e.g., about 10 wt % of the acrylate monomer, especially in cases in which a large amount (e.g., about 25 wt % or more) of functional hard monomer(s) are included, such as when more than about 5 wt % acidic hard functional monomers and 10 or more wt % (e.g., about 10-25 wt %) hydroxyl functional hard monomer(s) are included in the acrylate polymer.

Particularly useful are polar polyacrylates for holding a large amount of galantamine, such as polyacrylates having at least about 10 wt %, preferably at least about 20 wt %, preferably at least about 30 wt % acrylic monomers having hydroxyl group, acid group, or a combination thereof. One example is a polyacrylate having about 30 wt % hydroxyl group containing (—OH) monomer and about 3 wt % acid containing (—COOH) monomer. Another contains about 26 wt % —OH monomer and about 6 wt % —COOH monomer. Another useful polar polyacrylate contains about 10 wt % —OH monomer. Yet another useful polar polyacrylate contains about 20 wt % —OH monomer. The preferred —OH monomer is hydroxyethyl acrylate. The preferred —COOH monomer is acrylic acid.

Below is a table showing the T_{g}'s of exemplary soft and hard homopolymers the monomers of which are useful for making proadhesive of the present invention. Some of the monomers (e.g., acrylic acid, hydroxyethyl acrylate) are also functional monomers.

| | |
|---|---|
| poly(hydroxyethyl acrylate) (hard/functional monomer) | around 100° C. |
| poly(acrylic acid) (hard/functional monomer) | 106° C. |
| poly(vinyl acetate) (hard monomer) | 30° C. |
| poly(ethylhexyl acrylate) (soft monomer) | −70° C. |
| poly(isopropyl acrylate) (soft monomer) | −8° C. |
| poly(n-propyl acrylate) (soft monomer) | −52° C. |
| poly(isobutyl acrylate) (soft monomer) | −40° C. |
| poly(n-butyl acrylate) (soft monomer) | −54° C. |
| poly(n-octyl acrylate) (soft monomer) | −80° C. |

It has been found that the soft monomers 2-ethylhexyl acrylate and butyl acrylate are especially suitable to polymerize with functional monomers hydroxyethyl acrylate or acrylic acid either alone or in combination to form the acrylate polymer of the present invention. Further, the hard monomer vinyl acetate has been found to be very useful to polymerize with the soft monomers 2-ethylhexyl acrylate and butyl acrylate, either alone or in combination to form the proadhesive. Thus, the acrylate proadhesive polymer of the present invention is especially suitable to be made from 2-ethylhexyl acrylate or butyl acrylate copolymerized with hydroxyethyl acrylate, acrylic acid, or vinyl acetate, either alone or in combination. Another preferred hard monomer is t-octyl acrylamide, which can be used alone or in combination with other hard monomers such as acrylic acid and hydoxyethyl acrylate.

In an embodiment, the proadhesive is made by polymerizing monomers including about 30 to 75 wt % vinyl acetate, about 10-40 wt % hydroxyl functional monomer and about 10-70 wt % soft monomer such as 2-ethylhexyl acrylate or butyl acrylate. In a preferred embodiment, the proadhesive is made by polymerizing monomers including about 50 to 60 wt % vinyl acetate, about 10-20 wt % hydroxyethyl acrylate, and about 20-40 wt % 2-ethylhexyl acrylate. In some cases, no carboxyl (acid) group is used. Hydroxyethyl acrylate or hydroxypropyl acrylate can be used to provide hydroxyl functionality. For example, one embodiment is a proadhesive having about 50 wt % vinyl acetate, about 10 wt % hydroxyethyl acrylate, and about 40 wt % 2-ethylhexyl acrylate. As used herein, when a specific percentage is mentioned, it is contemplated there may be slight variations, e.g., of plus or minus 5% of the specific percentage (i.e., about 10 wt % may included 10 wt %±0.5 wt %). One other embodiment is a proadhesive having about 60 wt % vinyl acetate, about 20 wt % hydroxyethyl acrylate, and about 20 wt % 2-ethylhexyl acrylate.

In another embodiment, the proadhesive is made by polymerizing monomers including both monomer with hydroxyl group and monomer with carboxyl group. For example, certain preferred monomer combination for polymerization include an alkyl acrylate, an acrylamide, a monomer with hydroxyl group and a monomer with carboxyl group, e.g., making a proadhesive by polymerizing butyl acrylate, 2-hydroxyethyl acrylate or 2 hydroxypropyl acrylate or hydroxypropyl methacrylate, t-octyl acrylamide, and acrylic acid. In an embodiment, greater than 3 wt % of a hydroxypropyl acrylate or hydroxylpropyl methacrylate is used in making the acrylate polymer.

Acrylate polymers with high acid functionality (such as 5 wt % or more of acid monomers) is especially useful to delivery of galantamine. In certain cases for making a proadhesive in which both monomers with hydroxyl groups and monomer with carboxyl groups are to be polymerized with a soft monomer, e.g., butyl acrylate, the monomer proportions in the polymerization includes about 55 to 65 wt % soft monomer (e.g., butyl acrylate), about 5 to 15 wt % t-octyl acrylamide, about 20 to 30 wt % hydroxyethyl or hydroxypropyl acrylate and about 5 to 10 wt % acid monomer such as acrylic acid. In one embodiment, the acrylate polymer includes about 59 wt % butyl acrylate, about 10 wt % t-octyl acrylamide, about 25 wt % hydroxypropyl acrylate and about 6 wt % acrylic acid. In another embodiment, the hydroxypropyl acrylate is replaced with hydroxyethyl acrylate. In a preferred embodiment, when using acrylic acid to achieve the acidic and “hard” property, no vinyl acetate monomer is used in the polymerization of the acrylate polymer and there is no cross-linking.

It is desirable that with the incorporation of a large amount of permeation enhancers, the T_{g} of the resulting reservoir (with the drug, permeation enhancers and other ingredients) is such that the resulting reservoir would have good PSA properties for application to the body surface of an individual. Further, the resulting reservoir should not have cold flow that affects the normal application of the transdermal delivery. The acrylate polymer (or a blend of acrylate polymers) constitutes preferably about 40 wt % to 90 wt %, more preferably about 45 wt % to 80 wt % of the reservoir. It is possible to load drug and/or enhancer into the polymer composition to a high concentration, e.g., at or greater than about 20 dry weight %, at or greater than about 30 dry weight % (or solids wt %), even up to about 40 to 50 wt %, without adversely impacting the adhesion and rheological characteristics for pressure sensitive adhesive (PSA) application.

Preferred acrylate polymers or blends thereof provide the acrylic pressure sensitive properties in the delivery system glass transition temperature of about −10 to −40° C., preferably about −20 to −30° C. at application on a surface. The T_{g} of an acrylate polymer can be determined by differential scanning calorimetry (DSC) known in the art. Also, theoretical ways of calculating the T_{g} of acrylate polymers are also known. Thus, one having a sample of an acrylate polymer will be able to experimentally determine the T_{g}, for example, by DSC. One can also determine the monomer composition of the acrylate polymer and estimate theoretically the T_{g} by calculation. From the knowledge of the monomer composition of an acrylate polymer having galantamine and enhancer(s), one can also make the acrylate polymer without drug and enhancer and determine the T_{g}. According to the present invention, the acrylate materials, before dissolving the drug(s), permeation enhancers, etc., have T_{g}'s that are in the range of about −20 to 10° C., and have rheological properties that are not quite suitable for use directly as a PSA to skin because of the stiffness of the material. The acrylate polymers preferably have a molecular weight in a range of about 200,000 to 600,000. Molecular weight of acrylate polymers can be measured by gel permeation chromatography, which is known to those skilled in the art.

To control the physical characteristics of the acrylate polymer and the polymerization, it is preferred that monomers of molecular weight of below 500, even more preferably below 200 be used in the polymerization. Further, although optionally larger molecular weight monomers (linear macromonomers such as ELVACITE™ from ICI) can be used in the polymerization, it is preferred that they are not used. Thus, preferably no monomer of molecular weight (MW) above 5000, more preferably no monomer of MW above 2000, even more preferably no monomer of MW above 500, is to be included in the polymerization to form the acrylate polymer. Thus, in an aspect of the present invention, preferably, proadhesive polymers can be formed without macromonomers, or substantially without macromonomers, to have adhesive properties too stiff for PSA as is without incorporation of a large amount of permeation enhancers and drug. However, such proadhesives will become suitable for adhering to the skin as PSA in patch application after the appropriate amount of permeation enhancers and drug are dissolved therein.

However, if desired, in certain embodiments, optionally, the reservoir can include diluent materials capable of reducing quick tack, increasing viscosity, and/or toughening the reservoir structure, such as polybutylmethacrylate (ELVACITE, manufactured by ICI Acrylics, e.g., ELVACITE 1010, ELVACITE 1020, ELVACITE 20), polyvinylpyrrolidone, high molecular weight acrylates, i.e., acrylates having an average molecular weight of at least 500,000, and the like.

The acrylate polymers of the present invention can dissolve a large amount of permeation enhancer and allow the resulting drug and permeation enhancer-containing adhesive to have the desired adhesive and cohesive property without the drug or permeation enhancer separating out of the acrylate polymer matrix either as crystals or as oil. The resulting composition will be in the T_{g} and compliance range that it can be applied to a body surface without leaving an undesirable amount of residue material on the body surface upon removal of the device. The preferred acrylate polymer is not cross-linked. It is contemplated, however, that if desired, a nonsubstantial amount of cross-linking may be done, so long as it does not change substantially the T_{g}, creep compliance and elastic modulus of the acrylate polymer. It is also found that higher T_{g} and higher molecular weight of the acrylate are important for the acrylate polymer tolerating high enhancer loading. Since the measurement of the molecular weight of an acrylate polymer is difficult, precise or definite values are often not obtainable. More readily obtainable parameters that are related to molecular weight and drug and enhancer tolerance (i.e., solubility) are creep compliance and elastic modulus.

Enhancers typically behave as plasticizers to acrylate adhesives. The addition of an enhancer will result in a decrease in modulus as well as an increase in creep compliance, the effect of which is significant at high enhancer loading. A high loading of enhancers will also lower the T_{g} of the acrylate polymer. Thus, to achieve a proadhesive that is tolerant of high enhancer loading, other than increasing the T_{g} by using a higher ratio of hard monomer to soft monomer and the selection of suitable monomers, it is desirable to provide suitable higher molecular weight such that chain entanglement would help to achieve the desirable rheology. As a result, selecting a higher T_{g} and higher molecular weight for a proadhesive will increase the elastic modulus and decrease the creep compliance of the acrylate, making the proadhesive more enhancer tolerant. The measurement of the molecular weight of an acrylate polymer is often method-dependent and is strongly affected by polymer composition, since acrylate polymers discussed here are mostly copolymers, not homopolymers. More readily obtainable parameters that relate to molecular weight and drug and enhancer tolerance (i.e., solubility) are creep compliance and elastic modulus.

According to the present invention, especially useful polymeric materials for forming drug-containing PSA are acrylate polymers that, before the incorporation of drugs, enhancers, etc., and other ingredients for transdermal formation, have creep compliance (measured at 30° C. and 3600 second) of about 7×10⁻⁵ cm²/dyn or below and storage modulus G′ about 8×10⁵ dyn/cm² or above. Preferably the creep compliance is about 6×10⁻⁵ cm²/dyn to 2×10⁻⁶ cm²/dyn, more preferably about 5×10⁻⁵ cm²/dyn to 4×10⁻⁶ cm²/dyn. Preferably the storage modulus is about 8×10⁵ dyn/cm² to 5×10⁶ dyn/cm², more preferably about 9×10⁵ dyn/cm² to 3×10⁶ dyn/cm². Such creep compliance and modulus will render these acrylate polymers too stiff and unsuitable “as is” for dermal PSA applications. However, it was found that after formulating into a transdermal system with drugs, permeation enhancers, and the like, which produce plasticizing effect as well as tackifying effect, the acrylate polymers plasticized with permeation enhancers and/or drug would have a desirable storage modulus and creep compliance that are suitable for transdermal PSA applications. For example, the plasticized material would have a resulting creep compliance that is about 1×10⁻³ cm²/dyn or less, preferably more than about 7×10⁻⁵ cm²/dyn, preferably from about 7×10⁻⁵ cm²/dyn to 6×10⁴ cm²/dyn, more preferably about 1×10⁻⁴ cm²/dyn to 6×10⁻⁴ cm²/dyn. The preferred storage modulus of the plasticized acrylate polymer is about 1×10⁵ dyn/cm² to 8×10⁵ dyn/cm², preferably about 1.2×10⁵ dyn/cm² to 6×10⁵ dyn/cm², more preferably about 1.4×10⁵ dyn/cm² to 5×10⁵ dyn/cm².

It was found that incorporating the proper selection of drug and other ingredient (such as permeation enhancers) and using appropriate amounts thereof can change the T_{g}, storage modulus G′, and creep compliance sufficiently to result in an effective transdermal drug delivery system with the right adhesive properties for the desirable length of time, such as 24 hours, 3 day, or even 7 day application on a body surface. Such transdermal drug delivery systems will have little or no cold flow. As used herein, “little cold flow” means that any shape change of the device caused by cold flow is not noticeable by an average person on which the device is applied over the time of use. Particularly useful for forming adhesives incorporating an increased amount of beneficial agents (including drugs and permeation enhancers) over prior adhesives in transdermal drug delivery are the acrylic formulations containing a relatively lower percentage of soft monomers. It has been found that increasing the molecular weight increases the modulus of elasticity and decreases the polymer chain mobility via chain entanglements. Also, increasing hard monomer content increases the glass transition temperature.

Permeation enhancers can be useful for increasing the skin permeability of the drug galantamine or drug combinations to achieve delivery at therapeutically effective rates. Such permeation enhancers can be applied the skin by pretreatment or currently with the drug, for example, by incorporation in the reservoir. A permeation enhancer should have the ability to enhance the permeability of the skin for one, or more drugs or other biologically active agents. A useful permeation enhancer would enhance permeability of the desired drug or biologically active agent at a rate adequate for therapeutic level from a reasonably sized patch (e.g., about 20 to 80 cm²). Some useful permeation enhancers include non-ionic surfactant, one or more can be selected from the group including glyceryl mono-oleate, glyceryl mono-laurate, sorbitan mono-oleate, glyceryl tri-oleate, and isopropyl myristate. The non-ionic surfactant can be used in the amount of 0.1 about 25 wt % solids to the total composition of the matrix layer. Examples of permeation enhancers include, but are not limited to, fatty acid esters of alcohols, including fatty acid esters of glycerin, such as capric, caprylic, dodecyl, oleic acids; fatty acid esters of isosorbide, sucrose, polyethylene glycol; caproyl lactylic acid; laureth-2; laureth-2 acetate; laureth-2 benzoate; laureth-3 carboxylic acid; laureth-4; laureth-5 carboxylic acid; oleth-2; glyceryl pyroglutamate oleate; glyceryl oleate; N-lauroyl sarcosine; N-myristoyl sarcosine; N-octyl-2-pyrrolidone; lauraminopropionic acid; polypropylene glycol-4-laureth-2; polypropylene glycol-4-laureth-5dimethy-1 lauramide; lauramide diethanolamine (DEA). Preferred enhancers include, but are not limited to, pyroglutamate (such as octyl-, ethyl-, lauryl pyroglutamate (LP)), glyceryl monolaurate (GML), glyceryl monocaprylate, glyceryl monocaprate, glyceryl monooleate (GMO) and sorbitan monolaurate. Other permeation enhancers that could improve drug permeability include: isosorbide, oleth-4, ethoxydiglycol, and lauryl pyrrolidone. Additional examples of suitable permeation enhancers are described, for example, in U.S. Pat. Nos.: 5,785,991; 5,843,468; 5,882,676; and 6,004,578.

In some embodiments, especially some in which the reservoir does not necessarily have adequate adhesive property and a separate adhesive layer is used, a dissolution assistant can be incorporated in the reservoir to increase the concentration of the drug or biologically active ingredient within the reservoir layer. As for the dissolution assistant, one or more can be selected from the group including triacetin, isopropyl alcohol, propylene glycol, dimethylacetamide, propylene carbonate, diethylethanolanine, diethyl amine, triethylarnine, N-methyl morphorine and benzyamrnonium chloride, small acids such as lauric acid (lauryl acid), oleic acid, etc. Permeation enhancers can also act as solubization assistants. Non-ionic surfactants and dissolution assistants can be used in combination to increase the delivery rate of galantamine. As used herein, “permeation enhancers” is meant to include dissolution assistants, unless specified otherwise in context.

Also, in certain embodiments, the formulations can contain various types of enhancers. The first type is acidic, including, e.g., oleic acid, linoleic acid, linolenic acid, arachidonic acid, lauric acid, myristic acid, palmitic acid, carpric acid, myristoleic acid, palmitoleic acid, pidolic acid, N-lauroyl sarcosine, N-oleoyl Sarcosine, 2-hydroxy caprylic acid, serve as solubilizers for the polar galantamine, increasing the concentration of drug that can be loaded into the adhesive. The second set of useful enhancers are fatty acid esters, alcohol, or fatty acid/base reaction products, such as isopropyl myristate, isopropyl palmitate, lauryl pyrrolidone, laureth-2 laureth-4 glycerol monooleate glycerol monolaurate sorbitan monooleate sorbitan monolaurate lauryl lactate, 1,2-dihydroxydodecane, ethyl palmitate, PEG 200 monolaurate, Dioctylphthalate, and ethyl oleate. These can also serve as cosolvents for galantamine in skin, enabling high flux. When formulated together, the therapeutic dose can be delivered while remaining below the solubility limit. In addition, the formulations had low irritation potential on hairless guinea pigs and reasonable cohesive strength. In an embodiment, it has been found preferably at least two of the group consisting of oleic acid, lauric acid, and lauryl pyrrolidone, more preferably all three, are used together as permeation enhancers in the acrylate reservoir for delivery of galantamine.

In some embodiments, a large amount of permeation enhancer may be needed to aid the drug in transdermal delivery. The present invention is especially suitable for such transdermal delivery systems. Permeation enhancers in the polymer composition can be 10 wt % or high, 15 wt % and higher, 18 wt % and higher, greater than 20 wt %, or even greater than 30 dry weight % (or solids wt %). For effective delivery of galantamine, it has been found that a ratio of the amount (in wt %) of galantamine to amount of permeation enhancer (or a plurality of enhancers) of 1:2 to 2:1 is preferred. The permeation enhancers and the galantamine can constitute more than 25 wt % of the matrix reservoir, preferably more than 30 wt %, more preferably about 30 wt % to 50 wt %, in some embodiments preferably about 40 wt % to 50 wt % of the matrix reservoir.

In certain embodiments, polyvinylpyrrolidone (PVP) can be incorporated into the acrylate polymer matrix to increase cohesive strength and to affect the adhesive properties of the galantamine transdermal system. The incorporation of PVP resulted in an increase in modulus and decrease in creep compliance. PVP works particularly well with acrylate polymer adhesives, such as DURO-TAK® 87-201A, that contain hydroxyl or acid functionalities, or both. Both of these functionalities have the capability to interact with PVP. More than 5 wt %, preferably from 5 wt % to 20 wt % of PVP on matrix solids can be used to increase modulus, decrease creep compliance, and improve the adhesive properties of the transdermal formulation without reducing galantamine solubility significantly.

As aforementioned, the reservoir 3 contains galantamine, preferably totally dissolved in the matrix of the reservoir. It is understood that the reservoir can also contain other drugs, preferably in a single phase polymeric composition, free of undissolved components. Other drugs that can be contained in the drug reservoir include, for example, those disclosed in U.S. Pat. No. 6,004,578. One skilled in the art will be able to incorporate such drugs based on the disclosure of the present invention.

One or more permeation enhancers, alone or in combination, and which may include dissolution assistants, can constitute about 5 to 40% by weight, preferably about 10 to 35% by weight, and more preferably about 15 to 30% by weight solids of the resulting reservoir that has adequate pressure sensitive adhesive properties.

In certain embodiments, optionally, certain other plasticizer or tackifying agent is incorporated in the polyacrylate composition to improve the adhesive characteristics. Examples of suitable tackifying agents include, but are not limited to, aliphatic hydrocarbons; aromatic hydrocarbons; hydrogenated esters; polyterpenes; hydrogenated wood resins; tackifying resins such as ESCOREZ, aliphatic hydrocarbon resins made from cationic polymerization of petrochemical feedstocks or the thermal polymerization and subsequent hydrogenation of petrochemical feedstocks, rosin ester tackifiers, and the like; mineral oil and combinations thereof. The tackifying agent employed should be compatible with the polymer or blend of polymers.

Transdermal delivery patches typically have protective layers. For example, as shown in FIGS. 1 and 2, the patch 1 further includes a peelable protective layer 5. The protective layer 5 is made of a polymeric material that may be optionally metallized. Examples of the polymeric materials include polyurethane, polyvinyl acetate, polyvinylidene chloride, polypropylene, polycarbonate, polystyrene, polyethylene, polyethylene terephthalate, polybutylene terephthalate, paper, and the like, and a combination thereof. In preferred embodiments, the protective layer includes a siliconized polyester sheet.

The backing layer 2 may be formed from any material suitable for making transdermal delivery patches, such as a breathable or occlusive material including fabric or sheet, made of polyvinyl acetate, polyvinylidene chloride, polyethylene, polyurethane, polyester, ethylene vinyl acetate (EVA), polyethylene terephthalate, polybutylene terephthalate, coated paper products, aluminum sheet and the like, or a combination thereof. In preferred embodiments, the backing layer includes low density polyethylene (LDPE) materials, medium density polyethylene (MDPE) materials or high density polyethylene (HDPE) materials, e.g., SARANEX (Dow Chemical, Midland, Mich.). The backing layer may be a monolithic or a multilaminate layer. In preferred embodiments, the backing layer is a multilaminate layer including nonlinear LDPE layer/linear LDPE layer/nonlinear LDPE layer. The backing layer can have a thickness of about 0.012 mm (0.5 mil) to 0.125 mm (5 mil); preferably about 0.025 mm (1 mil) to 0.1 mm (4 mil); more preferably about 0.0625 mm (1.5 mil) to 0.0875 mm (3.5 mil).

Transdermal flux can be measured with a standard procedure using Franz cells or using an array of formulations. Flux experiments were done on isolated human cadaver epidermis. With Franz cells, in each Franz diffusion cell a disc of epidermis is placed on the receptor compartment. A transdermal delivery system is placed over the diffusion area (1.98 cm²) in the center of the receptor. The donor compartment is then added and clamped to the assembly. At time 0, receptor medium solution (between 21 and 24 ml, exactly measured) is added into the receptor compartment and the cell maintained at 35° C. This temperature yields a skin surface temperature of 30-32° C. Samples of the receptor compartment are taken periodically to determine the skin flux and analyzed by HPLC. In testing flux with an array of transdermal miniature patches, formulations are prepared by mixing stock solutions of each of the mixture components of formulation in organic solvents (about 15 wt % solids), followed by a mixing process. The mixtures are then aliquoted onto arrays as 4-mm diameter drops and allowed to dry, leaving behind solid samples or “dots.” (i.e., mini-patches). The miniature patches in the arrays are then tested individually for skin flux using a permeation array, whose principle of drug flux from a formulation patch through epidermis to a compartment of receptor medium is similar to that of Franz cells (an array of miniature cells). The test array has a plurality of cells, a piece of isolated human epidermis large enough to cover the whole array, and a multiple well plate with wells acting as the receptor compartments filled with receptor medium. The assembled permeation arrays are stored at 32° C. and 60% relative humidity for the duration of the permeation experiments. Receptor fluid is auto-sampled from each of the permeation wells at regular intervals and then measured by HPLC for flux of the drug.

A wide variety of materials that can be used for fabricating the various layers of the transdermal delivery patches according to this invention have been described above. It is contemplated that the use of materials other than those specifically disclosed herein, including those that may hereafter become known to the art to be capable of performing the necessary functions is practicable.

Administration of the Drug

On application to the skin, the drug in the drug reservoir of the transdermal patch diffuses into the skin where it is absorbed into the bloodstream to produce a systemic therapeutic effect. The onset of the therapeutic depends on various factors, such as, potency of the galantamine, the solubility and diffusivity of the drug in the skin, thickness of the skin, concentration of the drug within the skin application site, concentration of the drug in the drug reservoir, and the like. On repeated sequential applications (by replacing a used patch with a new one), the residual drug in the application site of the patch is absorbed by the body at approximately the same rate that drug from the new patch is absorbed into the new application area.

Administration of a patch can be maintained for a few days, e.g., at least three days, and up to 7 days.

Methods of Manufacture

The transdermal devices are manufactured according to known methodology. For example, in an embodiment, a solution of the polymeric reservoir material, as described above, is added to a double planetary mixer, followed by addition of desired amounts of the drug, permeation enhancers, and other ingredients that may be needed. Preferably, the polymeric reservoir material is an acrylate material. The acrylate material is solubilized in an organic solvent, e.g., ethanol, ethyl acetate, hexane, and the like. The mixer is then closed and activated for a period of time to achieve acceptable uniformity of the ingredients. The mixer is attached by means of connectors to a suitable casting die located at one end of a casting/film drying line. The mixer is pressurized using nitrogen to feed solution to the casting die. Solution is cast as a wet film onto a moving siliconized polyester web. The web is drawn through the lines and a series of ovens are used to evaporate the casting solvent to acceptable residual limits. The dried reservoir film is then laminated to a selected backing membrane and the laminate is wound onto the take-up rolls. In subsequent operations, individual transdermal patches are die-cut, separated and unit-packaged using suitable pouchstock. Patches are placed in cartons using conventional equipment. In another process, the drug reservoir can be formed using dry-blending and thermal film-forming using equipment known in the art. Preferably, the materials are dry blended and extruded using a slot die followed by calendering to an appropriate thickness.

### EXAMPLES

Below are examples of specific embodiments for carrying out the present invention. The examples are for illustrative purposes only, and are not intended to limit the scope of the present invention in any way. In the following examples all percentages are by weight unless noted otherwise. T_{g} was determined by DSC (Differential Scanning Calorimetry) with 10° C./min heating rate. Modulus G′ was storage modulus at 25° C. and 1 rad/s frequency (Frequency sweep experiment was conducted using AR-2000 rheometer from TA Instruments (TA Instruments, 109 Lukens Drive, New Castle, Del. 19720). The test conditions were: strain 1%, temperature 25° C., frequency range 0.1 to 100 rad/s, gap around 1000 micron). Creep compliance tests were conducted using AR-2000 rheometer from TA Instruments. The test conditions were: stress 1000 dyn/cm², temperature 30° C., time 3600 seconds, gap around 1000 microns. One skilled in the art will know how to measure T_{g}, creep compliance, and storage modulus in view of the present disclosure.

### Example 1

A monomer mix containing butyl acrylate, 2-hydroxyethyl acrylate, t-octyl acrylamide, acrylic acid, ethyl acetate (solvent), and 2,2′-azobisisobutyronitrile (AIBN) (polymerization initiator) was prepared. A fraction was charged to an appropriate vessel and heated to reflux with stirring. The remainder was added to the vessel over time. The ratios of the monomers and initiator added totally, i.e., butyl acrylate:2-hydroxyethyl acrylate:t-octyl acrylamide:acrylic acid:AIBN were 59:25.5:9.5:6:2. The material was then held at reflux for a suitable period of time. At the end of the hold period, the contents were cooled to room temperature and the solution polymer discharged. There was no cross-linking. The dry film made from this polyacrylate formulation had storage modulus of around 9×10⁵ dyn/cm², creep compliance of around 7×10⁻⁵ cm²/dyn, and glass transition temperature of −8° C., and consequently was too stiff to provide adequate adhesive properties alone. This formed a proadhesive.

### Example 2

A monomer mix containing butyl acrylate, 2-hydroxypropyl acrylate, t-octyl acrylamide, acrylic acid, ethyl acetate (solvent), and 2,2′-azobisisobutyronitrile (AIBN) (polymerization initiator) was prepared. A fraction was charged to an appropriate vessel and heated to reflux with stirring. The remainder was added to the vessel over time. The material was held at reflux for a suitable period of time. The ratios of the monomers and initiator added totally, i.e., butyl acrylate: 2-hydroxypropyl acrylate:t-octyl acrylamide:acrylic acid:AIBN were 59:25.5:9.5:6:2. At the end of the hold period, the contents were cooled to room temperature and the solution polymer discharged. There was no cross-linking. The dry film made from this polyacrylate formulation had storage modulus of around 8×10⁵ dyn/cm², creep compliance of around 4×10⁻⁵ cm²/dyn, and glass transition temperature of −8° C., and consequently was too stiff to provide adequate adhesive properties alone. This formed a proadhesive.

### Example 3

### Comparison of Galantamine Solubility

Adhesive films were prepared by mixing galantamine with the adhesive solution in ethyl acetate using the acrylate polymer of Example 2 to compare with the data for the adhesives in U.S. Pat. No. 5,700,480. Once a homogeneous mixture was formed, the adhesive and drug solution was cast on a PET/EVA release liner and dried at 65° C. for 90 minutes. The dried film was monitored for crystals over time using a cross-polarized microscope. The following Table 1 shows that in the present invention we were able to obtain more than 20 wt % of galantamine solubility in an acrylate polymer to have adequate adhesive properties, which would allow a transdermal delivery patch of reasonable, convenient-to-use reservoir thickness (e.g., less than 0.2 mm (8 mil) thick) and surface area, e.g., 48 cm², to be made, even for 7-day delivery. For comparison, the U.S. Pat. No. 5,700,480 galantamine formulation, due to the lower galantamine concentration, would require a thicker drug matrix.

TABLE 1
Galantamine solubility and adhesive thickness requirements

| | | Adhesive thickness |
|---|---|---|
| | | needed for 7-day patch |
| | Galantamine | with 80 cm² area |
| | Content | delivering 14.4 mg/day |
| Adhesive | (wt %) | mm (mil) |
| Acrylate Adhesive of U.S. Pat. | 10 | 0.25 (9.93) |
| 5,700,480 | | |
| Acrylate polymer of Example 2 | 23 | 0.11 (4.31) |

### Example 4

### The Comparison of Steady State Flux

Adhesive films were prepared by mixing galantamine and the permeation enhancers with a solution of the acrylate polymer of Example 2 in ethyl acetate. Once a homogeneous mixture was formed, the solution was cast on a release liner and dried at 65° C. for 90 minutes. The adhesive films were laminated to a PET/EVA backing layer and die-cut with an arch punch to a final diameter of 2.0 cm². The release liner was removed and the system was placed on the stratum corneum side of human cadaver epidermis mounted on the receptor side of the Franz cell. The donor and receptor sides of the Franz cell were clamped together and the receptor solution containing a phosphate buffer at pH 6.5 was added to the Franz cell. The cells were incubated in a shaker water bath at 35° C. for the duration of the experiment. Samples of the receptor solution were taken at regular intervals and the galantamine concentration is measured by HPLC. The removed receptor solution was replaced with fresh solution to maintain the sink conditions. Such flux measurement techniques were typical and well known by ones skilled in the art of transdermal drug delivery.

The galantamine flux in samples with galantamine and enhancers in the acrylate polymer of Example 2 were compared to that with an acrylate adhesive of the highest flux described in U.S. Pat. No. 5,700,480. The results in Table 2 show that we were able to achieve flux on human cadaver skin of greater than 10 μg/cm²hr. On the other hand, the flux on mice skin calculated according to the data of U.S. Pat. No. 5,700,480 was 2.7 μg/cm²hr. Since the U.S. Pat. No. 5,700,480 permeation experiments were done with mice skin, which is much more permeable than human skin, the permeation results of U.S. Pat. No. 5,700,480 are expected to be much lower than 2.7 μg/cm²hr if those experiments were done on human skin.

In this example of the present invention, the dried films were monitored for crystals over time using a cross-polarized microscope. It was found that there were no crystals, showing that the galantamine was completely dissolved in the matrix. It was also found that these polyacrylate formulations had desirable storage modulus and desirable creep compliance as shown in Table 3.

TABLE 2
Galantamine skin flux and crystal observations for various formulations

| | | | Primary | |
|---|---|---|---|---|
| | | | Irritation Index | |
| | Skin Flux | Skin Flux | (0.5 and | |
| | At Steady State | 0-168 hr | 48 hour | |
| Composition | (μg/cm²hr) | (μg/cm²hr) | score) | Crystals |
| Formulation of (U.S. Pat. | NA | 2.7 | N/A | None |
| 5,700,480)* | | | | |
| 18.5% Oleic Acid/1.5% Lauric | 9.23 | 8.93 | 1.7 | None |
| acid/ | | | | |
| 23.8% Galantamine/56.2% | | | | |
| acrylate of Example 2 | | | | |
| 8.3% Oleic Acid/5.5% Lauric | 11.35 | 11.35 | 2.6 | None |
| acid/ | | | | |
| 4.2% Lauryl Pyrrolidone/23.8% | | | | |
| Galantamine/58.2% acrylate of | | | | |
| Example 2 | | | | |
| 9% Oleic Acid/9% Lauryl | 7.67 | 6.71 | NA | None |
| Pyrrolidone/20% Galantamine/ | | | | |
| 62% acrylate of Example 2 | | | | |

*The permeation experiment was run with mice skin.

TABLE 3
Rheological properties for formulations in Table 2

| | | creep compliance |
|---|---|---|
| Adhesive # | Modulus (dyn/cm²) | (cm²/dyn) |
| 18.5% Oleic Acid/1.5% Lauric | 3.3 × 10⁵ | 2.1 × 10⁻⁴ |
| acid/ | | |
| 23.8% Galantamine/56.2% | | |
| acrylate of Example 2 | | |
| 8.3% Oleic Acid/5.5% Lauric | 2.6 × 10⁵ | 3.4 × 10⁻⁴ |
| acid/ | | |
| 4.2% Lauryl Pyrrolidone/23.8% | | |
| Galantamine/58.2% acrylate of | | |
| Example 2 | | |
| 9% Oleic Acid/9% Lauryl | 3.7 × 10⁵ | 1.7 × 10⁻⁴ |
| Pyrrolidone/20% Galantamine/ | | |
| 62% acrylate of Example 2 | | |

The primary skin irritation potential of a 7-day topical application of 5.07 cm² transdermal galantamine patch were evaluated on Guinea pigs. At the completion of the 7-day period the sites with the patches were scored for erytherma and edema at 30-40 minutes (nominal 0.5 hour), 23-25 hours (nominal 24 hours), and 47-49 hours (nominal 48 hours) after the test articles were removed. The 0.5 hour scores and the 48 hour scores were each averaged and Primary Irritation Index (PII) were calculated for each system as known in the art. An acceptable PII level is one that is less than 3.0. The results show that the samples of the present invention had acceptable Primary Irritation Indexes. A patch that does not result in irritation after one day of wear may cause irritation after a few days on the skin. Thus, acceptable Irritation Indexes for 7 day wear is an important parameter that affords advantages over shorter term patches.

### Example 5

### The Comparison of Steady State Flux

Adhesive films were prepared by mixing galantamine and the permeation enhancers with a solution of the acrylate polymer of Example 1 in ethyl acetate. Once a homogeneous mixture was formed, the solution was cast on a release liner and dried at 65° C. for 90 minutes. The adhesive films were laminated to a PET/EVA backing layer and die-cut with an arch punch to a final diameter of 2.0 cm². The release liner was removed and the system was placed on the stratum corneum side of human cadaver epidermis mounted on the receptor side of the Franz cell. The donor and receptor sides of the Franz cell were clamped together and the receptor solution containing a phosphate buffer at pH 6.5 was added to the Franz cell. The cells were incubated in a shaker water bath at 35° C. for the duration of the experiment. Samples of the receptor solution were taken at regular intervals and the galantamine concentration is measured by HPLC. The removed receptor solution was replaced with fresh solution to maintain the sink conditions.

The galantamine flux in samples with galantamine and enhancers in the acrylate polymer of Example 1 were compared to that with highest flux data for an acrylate adhesive described in U.S. Pat. No. 5,700,480. The results in Table 4 show that we were able to achieve human cadaver skin flux of greater than 3.5 μg/cm²hr. On the other hand, the flux on mice skin according to the data of U.S. Pat. No. 5,700,480 was lower, at 2.7 μg/cm²hr on mice skin.

In this example of the present invention, the dried films were monitored for crystals over time using a cross-polarized microscope. It was found that there were no crystals, showing that the galantamine was completely dissolved in the matrix. It was also found that these polyacrylate formulations had desirable storage modulus and desirable creep compliance as shown in Table 5.

TABLE 4
Galantamine skin flux and crystal observations for various formulations

| | Skin Flux | | Primary | |
|---|---|---|---|---|
| | At | Skin Flux | Irritation Index | |
| | Steady-State | 0-78 hr | (0.5 and 48 | |
| Composition | (μg/cm²hr) | (μg/cm²hr) | hour score) | Crystals |
| Adhesive of (U.S. Pat. | NA | 2.7 | N/A | None |
| 5,700,480)* | | | | |
| 8.3% Oleic Acid/5.5% Lauric acid/ | 5.13 | 3.70 | 1.9 | None |
| 4.2% Lauryl Pyrrolidone/24.4% | | | | |
| Galantamine/58.2% acrylate of | | | | |
| Example 1 | | | | |
| 18.5% Oleic Acid/1.5% Lauric | 4.61 | 3.55 | 2.4 | None |
| acid/23.8% Galantamine/ | | | | |
| 56.2% acrylate of Example 1 | | | | |

*The permeation experiment was run with mice skin.

TABLE 5
Rheological properties for formulations in Table 4

| | | creep compliance |
|---|---|---|
| Adhesive # | Modulus (dyn/cm²) | (cm²/dyn) |
| 8.3% Oleic Acid/5.5% Lauric | 2.1 × 10⁵ | 4.1 × 10⁻⁴ |
| acid/ | | |
| 4.2% Lauryl Pyrrolidone/24.4% | | |
| Galantamine/58.2% acrylate of | | |
| Example 1 | | |
| 18.5% Oleic Acid/1.5% Lauric | 2.8 × 10⁵ | 2.9 × 10⁻⁴ |
| acid/23.8% Galantamine/56.2% | | |
| acrylate of Example 1 | | |

The entire disclosure of each patent, patent application, and publication cited or described in this document is hereby incorporated herein by reference. The practice of the present invention will employ, unless otherwise indicated, conventional methods used by those in pharmaceutical product development within those of skill of the art. Embodiments of the present invention have been described with specificity. The embodiments are intended to be illustrative in all respects, rather than restrictive, of the present invention. It is to be understood that various combinations and permutations of various constituents, parts and components of the schemes disclosed herein can be implemented by one skilled in the art without departing from the scope of the present invention.

What is claimed is:

1. A device for transdermal administration of galantamine to an individual in need thereof, comprising a backing and a drug reservoir comprising acrylate polymer with polar functional monomer component, galantamine dissolved in the acrylate polymer at greater than 10 wt %, and permeation enhancer of sufficient amount to deliver the galantamine at a flux of greater than 4.5 μg/cm²-hr.

2. The device of claim 1 having greater than 15 wt % of permeation enhancer in the drug reservoir and wherein the flux is greater than 10 μg/cm²-hr transdermally.

3. The device of claim 1 wherein the drug reservoir has 15 wt % or more of galantamine and wherein galantamine together with permeation enhancer constitute greater than 30 wt % of the drug reservoir.

4. The device of claim 1 wherein the drug reservoir includes a permeation enhancer selected from the group consisting of lauric acid, ester of lauric acid, laureth-2, ester of laureth-2, glyceryl monooleate, lauryl pyrrolidone, laureth-4, oleic acid, linoleic acid, linolenic acid, arachidonic acid, myristic acid, isopropyl myristate, lauryl lactate, 1,2-dihydroxydodecane, ethyl palmitate, and N-lauroyl sarcosine.

5. The device of claim 1 wherein the drug reservoir includes a permeation enhancer selected from the group consisting of oleic acid, linoleic acid, linolenic acid, arachidonic acid, lauric acid, myristic acid, palmitic acid, carpric acid, myristoleic acid, palmitoleic acid, pidolic acid, N-lauroyl sarcosine, N-oleoyl Sarcosine, 2-hydroxy caprylic acid isopropyl myristate, isopropyl palmitate, lauryl pyrrolidone, laureth-2, laureth-4, glycerol monooleate, glycerol monolaurate, sorbitan monooleate, sorbitan monolaurate, lauryl lactate, 1,2-dihydroxydodecane, ethyl palmitate, PEG 200 monolaurate, Dioctylphthalate, ethyl oleate, and has greater than 15 wt % of galantamine.

6. The device of claim 1 wherein the drug reservoir includes oleic acid, lauric acid and lauryl pyrrolidone as permeation enhancers.

7. The device of claim 1 wherein the acrylate polymer has at least 10 wt % functional monomer content, constitutes 45 wt % to 80 wt % of the drug reservoir and has at least 30 wt % content of galantamine and permeation enhancer combination, the acrylate polymer having a T_{g} of greater than −15° C. if without permeation enhancer and without galantamine, the drug reservoir having pressure sensitive adhesive properties applicable to the body surface for transdermal delivery.

8. The device of claim 1 wherein the drug reservoir in the device includes permeation enhancer wherein the drug reservoir is of a composition having a creep compliance of 6×10⁻⁵ cm²/dyn to 2×10⁻⁶ cm²/dyn if the drug reservoir is without galantamine and without permeation enhancer.

9. The device of claim 1 wherein the acrylate polymer includes an acrylic copolymer having (i) 40 to 50 wt % of soft alkyl acrylate monomer component, in which each soft alkyl acrylate monomer having a homopolymer T_{g} of −80 to −20° C., (ii) at least 40 wt % of hard modifying monomer component which includes hard functional monomer, each hard modifying monomer having a homopolymer T_{g} of 0 to 250° C., and (iii) 10 to 35 wt % of functional monomer.

10. The device of claim 1 wherein the acrylate polymer has (i) 40 to 50 wt % of soft alkyl acrylate monomer component, in which each soft alkyl acrylate monomer having a homopolymer T_{g} of −80 to −20° C., (ii) 40 to 60 wt % of hard modifying monomer component, in which each hard modifying monomer having a homopolymer T_{g} of 0 to 250° C., and (iii) 10 to 35 wt % of functional monomer component, wherein soft monomer is an alkyl acrylate monomer having 4 to 10 carbon atoms in the alkyl group.

11. The device of claim 1 wherein the acrylate polymer includes a soft acrylate monomer selected from the group consisting of butyl, hexyl, 2-ethylhexyl, octyl, and dodecyl acrylates and isomers thereof.

12. The device of claim 1 wherein the acrylate polymer includes 40 to 50 wt % of soft alkyl acrylate monomer component having a homopolymer T_{g} of less than −20° C.

13. The device of claim 1 wherein the acrylate polymer includes 40 to 50 wt % of soft alkyl acrylate monomer component having a homopolymer T_{g} of less than −20° C., hard modifying monomer component having a homopolymer T_{g} of higher than 20° C., and functional monomer having acidic group.

14. The device of claim 1 wherein the acrylate polymer includes hard modifying monomer having a homopolymer T_{g} of 0 to 250° C., wherein the permeation enhancer and the galantamine are dissolved in the acrylate polymer and the acrylate polymer has a T_{g} of 0 to −20° C. and a creep compliance of 6×10⁻⁵ cm²/dyn to 2×10⁻⁶ cm²/dyn without dissolved galantamine and permeation enhancer, whereas the drug reservoir with the dissolved galantamine and permeation enhancer has a creep compliance of less than 1×10⁻³ cm²/dyn and storage modulus of 1×10⁵ dyn/cm² to 8×10⁵ dyn/cm.

15. The device of claim 1 wherein the acrylate polymer includes hard modifying monomer having a homopolymer T_{g} of 40 to 100° C.

16. The device of claim 1 wherein the acrylate polymer has acidic group and hydroxyl group therein and includes 5 to 15 wt % nonfunctional hard monomer.

17. The device of claim 1 wherein the acrylate polymer includes monomer components including 20 to 30 wt % hydroxyethyl or hydroxypropyl acrylate and 5 to 10 wt % acid monomer and without vinyl acetate.

18. The device of claim 1 wherein the acrylate polymer includes functional monomer selected from the group consisting of acrylic acid, hydroxyethyl acrylate, and hydroxypropyl acrylate.

19. The device of claim 1 wherein the permeation enhancer and the galantamine are dissolved in the acrylate polymer and the acrylate polymer has a T_{g} of 0 to −20° C., a creep compliance of 6×10⁻⁵ cm²/dyn to 2×10⁻⁶ cm²/dyn without dissolved galantamine and permeation enhancer, whereas with the dissolved galantamine and permeation enhancer the acrylate polymer forms a drug reservoir with a T_{g} of −10 to −20° C., a creep compliance of less than 1×10⁻³ cm²/dyn and storage modulus of 1×10⁵dyn/cm² to 8×10⁵ dyn/cm².

20. The device of claim 1 wherein the acrylate polymer has a T_{g} of 0 to −20° C. if without galantamine and permeation enhancer, whereas the acrylate polymer with galantamine and permeation enhancer at above 30 wt % in a single phase forms a drug reservoir with a T_{g} of −10 to −20° C., a creep compliance of 1×10 cm²/dyn to 6×10⁴ cm²/dyn and storage modulus of 1×10⁵dyn/cm² to 8×10⁵ dyn/cm².

21. The device of claim 1 wherein the acrylate polymer has a T_{g} of 0 to −20° C., storage modulus of 8×10⁵ dyn/cm² or above ° C. if without galantamine and permeation enhancer, whereas the acrylate polymer with galantamine and permeation enhancer at above 30 wt % forms a drug reservoir with a T_{g} of −10 to −40° C., a creep compliance of 1×10⁻⁴ cm²/dyn to 6×10⁻⁴ cm²/dyn and storage modulus of 1×10⁵ dyn/cm² to 8×10⁵ dyn/cm².

22. A device for transdermal administration of galantamine to an individual in need thereof, comprising a backing and a drug reservoir comprising 20 wt % or more of galantamine, 10 wt % or more of permeation enhancer content to deliver the galantamine at an flux of 10 μg/cm²-hr or more, at least one permeation enhancer selected from the group consisting of lauric acid, ester of lauric acid, laureth-2, ester of laureth-2, glyceryl monooleate, lauryl pyrrolidone, laureth-4, oleic acid, isopropyl myristate, lauryl lactate, 1,2-dihydroxydodecane, ethyl palmitate, and N-lauroyl sarcosine.

23. A method of making a drug reservoir for transdermal galantamine delivery, comprising: providing an acrylate polymer with functional monomer, incorporating galantamine and permeation enhancer in the acrylate polymer to form a drug reservoir with more than 10 wt % of galantamine dissolved in the drug reservoir such that the drug reservoir can deliver the galantamine at a flux of greater than 4.5 μg/cm²-hr, the acrylate polymer constitutes 45 wt % to 80 wt % of the drug reservoir, wherein the drug reservoir is a pressure sensitive adhesive applicable to the body surface.

24. The method of claim 23 comprising dissolving more than 15 wt % galantamine and dissolving permeation enhancer in the drug reservoir such that the galantamine and permeation enhancer make up greater than 30 wt % dissolved solids in the drug reservoir and wherein the flux is greater than 10 μg/cm²-hr.

25. The method of claim 23 comprising dissolving in the drug reservoir a permeation enhancer selected from the group consisting of lauric acid, ester of lauric acid, laureth-2, ester of laureth-2, glyceryl monooleate, lauryl pyrrolidone, laureth-4, oleic acid, linolenic acid, linoleic acid, arachidonic acid, palmitic acid, myristic acid, isopropyl myristate, lauryl lactate, 1,2-dihydroxydodecane, ethyl palmitate, and N-lauroyl sarcosine.

26. The method of claim 24 wherein the drug reservoir has a glass transition temperature T_{g} of less than −10° C. whereas the acrylate polymer has a T_{g} of greater than −15° C. and a creep compliance of 6×10⁻⁵ cm²/dyn to 2×10⁻⁶ cm²/dyn and the drug reservoir includes lauryl pyrrolidone and at least one of oleic acid and lauric acid as permeation enhancers.

27. The method of claim 24 wherein the acrylate polymer includes (i) 40 to 50 wt % of soft alkyl acrylate monomer component, in which each soft alkyl acrylate monomer having a homopolymer T_{g} of −80 to −20° C., (ii) 40 to 60 wt % of nonfunctional hard modifying monomer component, in which each hard modifying monomer having a homopolymer T_{g} of 0 to 250° C., and (iii) up to 30% by weight of functional monomer component, wherein the soft monomer is an alkyl acrylate monomer having 4 to 10 carbon atoms in the alkyl group.

28. The method of claim 24 wherein the acrylate polymer includes a soft acrylate monomer selected from the group consisting of butyl, hexyl, 2-ethylhexyl, octyl, and dodecyl acrylates and isomers thereof.

29. The method of claim 24 wherein the acrylate polymer includes 40 to 50 wt % of soft alkyl acrylate monomer component having a homopolymer T_{g} of less than −20° C.

30. The method of claim 24 wherein the acrylate polymer has a T_{g} of 0 to −20° C. if without galantamine and permeation enhancer, and the drug reservoir having the dissolved galantamine and permeation enhancer has a T_{g} of −10 to −20° C., a creep compliance of 1×10⁻⁴ cm²/dyn to 6×10⁻⁴ cm²/dyn and storage modulus of 1×10⁵ dyn/cm² to 8×10⁵ dyn/cm².

31. The method of claim 24 comprising incorporating permeation enhancer and galantamine in the acrylate polymer in single phase, wherein the acrylate polymer has a T_{g} of 0 to −20° C., storage modulus of 8×10⁵ dyn/cm² or above if without the galantamine and permeation enhancer, and the drug reservoir with the galantamine and permeation enhancer has a T_{g} of −10 to −20° C., a creep compliance of 1×10⁻⁴ cm²/dyn to 6×10⁻⁴ cm²/dyn and storage modulus of 1×10⁵ dyn/cm² to 8×10⁵ dyn/cm².

32. The method of claim 24 comprising providing the acrylate polymer having monomer components including 20 to 30 wt % hydroxyethyl or hydroxypropyl acrylate and 5 to 10 wt % acid monomer, but without vinyl acetate.

33. The method of claim 24 comprising providing the acrylate polymer having monomer components of 55 to 65 wt % butyl acrylate, 5 to 15 wt % t-octyl acrylamide, 20 to 30 wt % hydroxyethyl or hydroxypropyl acrylate and 5 to 10 wt % acid monomer.

34. The method of claim 23 comprising including 18 wt % or more of permeation enhancer in the drug reservoir.

35. The method of claim 23 wherein the device can deliver 14 to 21 mg galantamine per day and the area of the device contacting the skin is 80 cm² or less.

36. A method of making a transdermal galantamine delivery drug reservoir, comprising: providing for a drug reservoir a proadhesive of inadequate adhesive properties for removable adhesion to skin, the proadhesive containing functional monomer with acidic group and having a creep compliance of 6×10⁻⁵ cm²/dyn to 2×10⁻⁶ cm²/dyn, storage modulus of 8×10⁵ dyn/cm² or above and a creep compliance of below 7×10⁻⁵ cm²/dyn, incorporating galantamine and permeation enhancer combination in the proadhesive with a dissolved concentration of greater than 30 wt % solids of galantamine and permeation enhancer combination such that the resulting drug reservoir has adhesive properties appropriate for transdermal galantamine delivery, the resulting drug reservoir having a creep compliance of 1×10⁻⁴ cm²/dyn to 6×10⁻⁴ cm²/dyn and storage modulus of 1×10⁵ dyn/cm² to 8×10⁵ dyn/cm².
